(19) United States
(12) Patent Application Publication
(10) Pub. No.: US 2010/0125329 A1
(43) Pub. Date: May 20, 2010

(54) Pseudoelastic stents having a drug coating and a method of producing the same

(76) Inventors: Zhi Cheng Lin, Mountain View, CA (US); Winnette McIntosh, San Jose, CA (US)

Correspondence Address: WORKMAN NYDEGGER, 1000 EAGLE GATE TOWER,, 60 EAST SOUTH TEMPLE, SALT LAKE CITY, UT, 84111, US

(21) Appl. No.: 10/327,371

(22) Filed: Dec. 19, 2002

Related U.S. Application Data
(63) Continuation-in-part of application No. 09/896,435, filed on Jun. 29, 2001, now Pat. No. 6,602,272.
Application No. 09/896,435 is a continuation-in-part of application No. 09/705,422, filed on Nov. 2, 2000.

(51) Int. Cl. A61F 2/82; B05D 3/06; B05D 3/10

(52) U.S. Cl. 623/1.42; 623/1.46; 427/2.25; 216/37

(57) ABSTRACT

An implantable medical device, such as a stent, having linear pseudoelastic behavior and a polymeric drug coating is disclosed. A method of producing an implantable medical device having linear pseudoelastic behavior and a polymeric drug coating is also disclosed.

### CROSS-REFERENCE

This is a continuation-in-part of application Ser. No. 09/896,435, which was filed on Jun. 29, 2001, which is a continuation-in-part of application Ser. No. 09/705,422, which was filed on Nov. 2, 2000.

### BACKGROUND OF THE INVENTION

1. Field of the Invention

This invention is directed to an implantable medical device, such as a stent, having linear pseudoelastic behavior and a polymeric drug coating, and method of forming the same.

2. Description of the Background

Percutaneous transluminal coronary angioplasty (PTCA) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across the occlusive lesion. Once in position across the lesion, the balloon is inflated to a predetermined size to remodel the vessel wall. The balloon is then deflated to a smaller profile to allow the catheter to be withdrawn from the patient's vasculature.

A problem associated with the above procedure includes formation of intimal flaps or torn arterial linings, which can collapse and occlude the conduit after the balloon is deflated. Vasospasms and recoil of the vessel wall also threaten vessel closure. Moreover, thrombosis and restenosis of the artery may develop over several months after the procedure, which may necessitate another angioplasty procedure or a surgical by-pass operation. To reduce the partial or total occlusion of the artery by the collapse of arterial lining and to reduce the chance of the development of thrombosis and restenosis, an expandable, intraluminal prosthesis, also known as a stent, is implanted in the lumen to maintain the vascular patency.

Stents act as scaffoldings, functioning to physically hold open and, if desired, to expand the wall of the passageway. Typically, stents are capable of being compressed so that they can be inserted through small lumens via catheters and then expanded to a larger diameter once they are at the desired location. Mechanical intervention via stents has reduced the rate of restenosis as compared to balloon angioplasty. Yet, restenosis is still a significant clinical problem with rates ranging from 20-40%. When restenosis does occur in the stented segment, its treatment can be challenging, as clinical options are more limited as compared to lesions that were treated solely with a balloon.

Stents are used not only for mechanical intervention but also as vehicles for providing biological therapy. Biological therapy can be achieved by medicating the stents. Medicated stents provide for the local administration of a drug at the diseased site. In order to provide an efficacious concentration to the treated site, systemic administration of such medication often produces adverse or even toxic side effects for the patient. Local drug delivery is a preferred method of treatment in that smaller total levels of medication are administered in comparison to systemic dosages, but are concentrated at a specific site. Local drug delivery thus produces fewer side effects and achieves more favorable results.

One proposed method of medicating stents involves the use of a polymeric carrier coated onto the surface of the stent. A composition including a solvent, a polymer dissolved in the solvent, and a drug dispersed in the blend is applied to the stent by immersing the stent in the composition or by spraying the composition onto the stent. The solvent is allowed to evaporate, leaving on the stent surfaces a coating of the polymer and the drug impregnated in the polymer.

A potential shortcoming of conventional medicated stents is that the polymeric drug coating can be damaged when the stent is processed for use. For example, the polymeric drug coating can be damaged when the coated stent is collapsed in order to be placed onto a delivery device (e.g., catheter). Some conventional drug-eluting stents have a metallic stent body formed by a self-expanding superelastic material such as nitinol. One of the processes used to collapse a self-expanding nitinol stent is to subject the stent to a temperature below the martensitic finish temperature of the nitinol material. In order to obtain 100% martensite by a thermal treatment process, the temperature used to collapse the stent has to be as low as −100° C. Many of the polymers that might be used on a drug-eluting stent are very brittle at these low temperatures, and therefore the polymeric drug coatings are susceptible to cracking during the stent manufacturing process.

Accordingly, what is needed is a stent that addresses the aforementioned drawback.

### SUMMARY

In accordance with one aspect of the present invention, an implantable medical device for insertion into a biological lumen is disclosed, comprising a metallic body including a linear pseudoelastic material, and a coating including a polymer disposed over a portion of the body. In one embodiment, the linear pseudoelastic material does not undergo phase transformation at room temperature when the body is subjected to stress. In another embodiment, the coating further includes a therapeutic substance.

In accordance with another aspect, an implantable medical device for insertion into a biological lumen is disclosed, comprising a metallic substrate and a coating including a polymer disposed over a portion of the substrate, wherein the substrate is in a martensitic phase when the substrate is stressed into a first shape and the substrate remains in a martensitic phase when the stress on the substrate is relieved. In one embodiment, a stress-strain hysteresis curve for the substrate does not include a stress plateau.

In yet another aspect of the present invention, a stent for insertion into a biological lumen is disclosed, comprising a self-expanding body including a cold formed nickel-titanium alloy that exhibits linear pseudoelasticity, and a coating disposed over a portion of the body, the coating including a polymer and optionally a therapeutic substance. In an embodiment, the cold formed nickel-titanium alloy comprises a cold worked percentage of about 30% to about 60%. In another embodiment, the nickel-titanium alloy is pseudoelastic when stressed without onset of stress-induced martensite.

In accordance with another aspect, a method of producing an implantable medical device is disclosed, comprising applying a coating having a polymer, and optionally a therapeutic substance, to a substrate, the substrate comprising a linear pseudoelastic material.

In a further aspect, a method of producing a drug-eluting stent having linear pseudoelastic behavior is disclosed, comprising forming struts by selectively removing portions of a tubular substrate, the substrate comprising a cold worked metallic material, and applying a coating to the struts, the coating comprising a polymer and a therapeutic substance. In an embodiment of the present invention, the formation of the struts is performed by using a low-energy laser. In another embodiment, the formation of the struts is performed by chemical etching.

### BRIEF DESCRIPTION OF THE DRAWINGS

FIG. 1 is a partial side view of a stent in one embodiment of the present invention;

FIG. 2 is a set of stress-strain curves for conventional 316L stainless steel, a linear pseudoelastic material, and a non-linear pseudoelastic material; and

FIGS. 3A, 3B, 4A and 4B are stress-strain hysteresis curves for a nickel-titanium alloy in accordance with Example 1.

### DETAILED DESCRIPTION OF THE EMBODIMENTS

### Implantable Medical Device

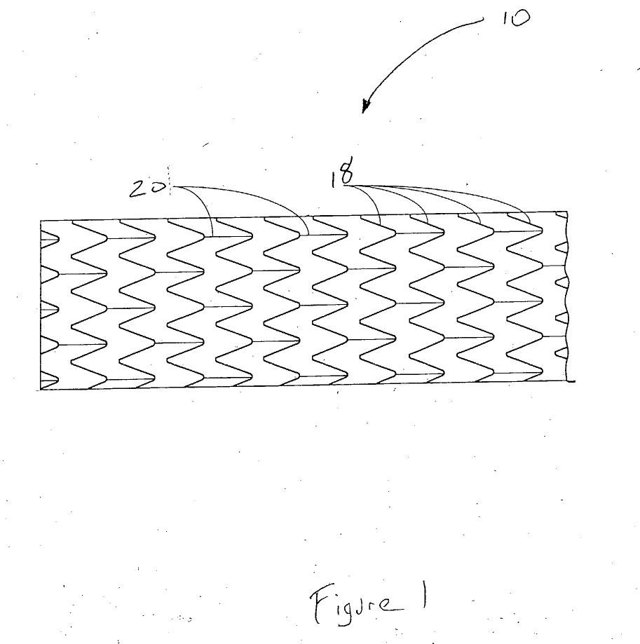

Referring to FIG. 1, stent 10 can have a tubular body of structural members including struts 18. Struts 18 are radially expandable and interconnected by connecting elements 20 that are disposed between adjacent struts 18. Both struts 18 and connecting elements 20 have an outer (or lumen contacting) surface and an inner surface. In an embodiment of the present invention, stent 10 has a metallic body that includes a material that demonstrates linear pseudoelastic behavior. In addition, at least a portion of the body is coated with a polymer and a therapeutic substance.

“Pseudoelasticity” is the capacity of a material to undergo large elastic strains when stressed and to substantially fully recover all strain upon removal of the stress. For example, near equi-atomic binary nickel-titanium alloys can exhibit “pseudoelastic” behavior and undergo strains on the order of 8 percent or more. “Substantial full recovery” is typically understood to be less than about 0.5 percent unrecovered strain, also known as permanent set or amnesia. Pseudoelasticity can be further divided into two subcategories: “linear” pseudoelasticity and “non-linear” pseudoelasticity. “Non-linear” pseudoelasticity is understood to be synonymous with “superelasticity.” Non-linear pseudoelasticity, in its idealized state, exhibits a relatively flat loading plateau in which a large amount of recoverable strain is possible with very little increase in stress. This flat plateau can be seen in the stress-strain hysteresis curve of the material. Linear pseudoelasticity exhibits no such flat plateau. Non-linear pseudoelasticity is known to occur due to a reversible phase transformation from austenite to martensite, the latter more precisely called “stress-induced martensite” (SIM). Linear pseudoelasticity has no such phase transformation associated with it. Further discussions of linear pseudoelasticity can be found in, for example, T. W. Duerig et al., “Linear Superelasticity in Cold-Worked Ni—Ti,” Engineering Aspects of Shape Memory Alloys, pp. 414-19 (1990).

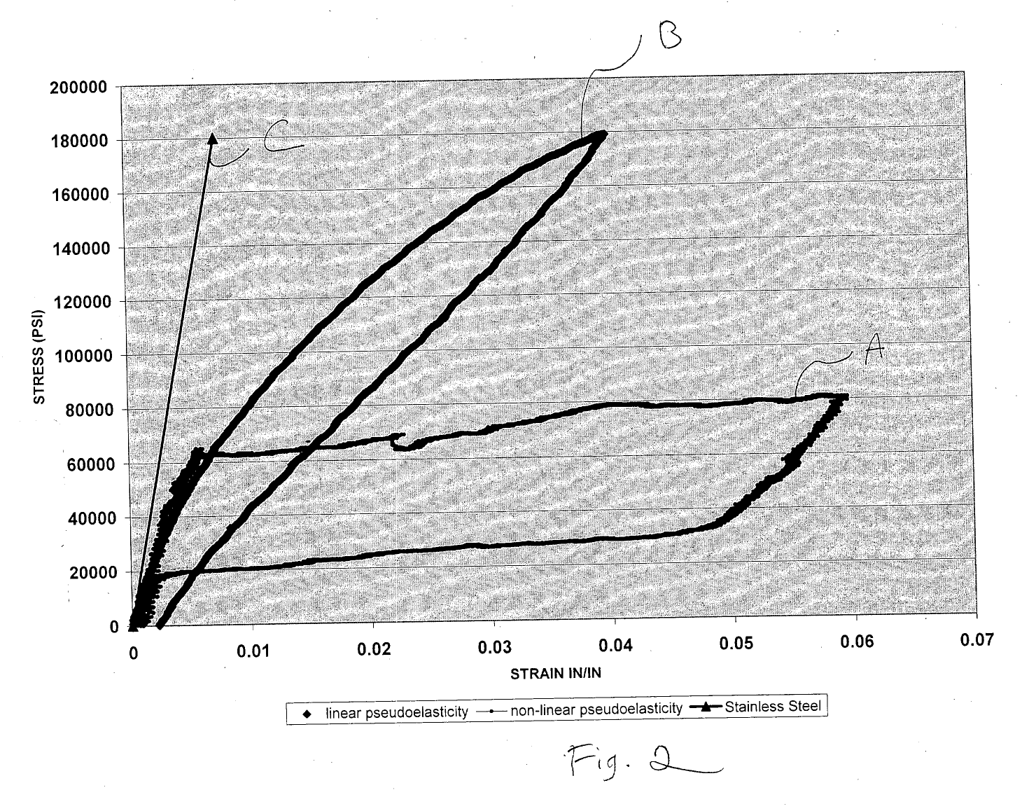

FIG. 2 illustrates an example of a stress-strain curve of a linear pseudoelastic material, as compared to 316L stainless steel and a non-linear pseudoelastic material. In an embodiment of the present invention, the structural members of stent 10 are formed partially or completely of a material that has linear pseudoelastic behavior as shown in FIG. 2.

In FIG. 2, curve A illustrates the strain/stress relationship of a non-linear pseudoelastic material. The x and y-axes are labeled in units of stress from zero to 200 ksi and strain from 0 to 7 percent, respectively. In curve A, when stress is applied to a specimen of a metal such a nitinol exhibiting non-linear pseudoelastic characteristics at a temperature at or above that which the transformation of the martensitic phase to the austenitic phase is complete, the specimen deforms elastically until it reaches a particular stress level where the alloy then undergoes a stress-induced phase transformation from the austenitic phase to the martensitic phase (i.e., the stress-induced martensite phase). As the phase transformation progresses, the material undergoes significant increases in strain with little or no corresponding increases in stress. On curve A this is represented by upper, nearly flat stress plateau at approximately 60 to 80 ksi. The strain increases while the stress remains essentially constant until the transformation of the austenitic phase to the martensitic phase is complete. Thereafter, further increase in stress is necessary to cause further deformation. The martensitic metal first yields elastically upon the application of additional stress and then plastically with permanent residual deformation (not shown).

If the load on the specimen is removed before any permanent deformation has occurred, the martensite specimen elastically recovers and transforms back to the austenitic phase. The reduction in stress first causes a decrease in strain. As stress reduction reaches the level at which the martensitic phase transforms back into the austenitic phase, the stress level in the specimen remains essentially constant (but less than the constant stress level at which the austenitic crystalline structure transforms to the martensitic crystalline structure until the transformation back to the austenitic phase is complete). In other words, there is significant recovery in strain with only negligible corresponding stress reduction. This is represented in curve A by the lower stress plateau at about 20 ksi.

After the transformation back to austenite is complete, further stress reduction results in elastic strain reduction. This ability to incur significant strain at relatively constant stress upon the application of a load and to recover from the deformation upon the removal of the load is commonly referred to as a non-linear pseudoelasticity (or superelasticity).

FIG. 2 also has a curve B representing the behavior of a linear pseudoelastic material as utilized in the present invention. Curve A generally has a higher slope or Young's Modulus than curve B. Also, curve B does not contain any flat plateau stresses found in curve A. This stands to reason since the material of curve B remains in the martensitic phase throughout and does not undergo any phase change. The same tension and release of stress cycle to generate curve A is used to generate curve B. To that end, curve B shows that increasing stress begets a proportional increase in reversible strain, and a release of stress begets a proportional decrease in strain. The areas bounded by curves A and B represent the hysteresis in the material.

As apparent from comparing curve B to curve A in FIG. 2, with the use of a linear pseudoelastic material, the mechanical strength of the present invention medical device is substantially greater per unit strain than a comparable device made of a superelastic material. Consequently, a major benefit is that smaller component parts such as struts 18 can be used because of the greater storage of energy available in a linear pseudoelastic device. A small profile is one critical factor for crossing narrow lesions or for accessing remote and tortuous arteries.

FIG. 2 also includes curve C which is the elastic behavior of a standard 316L stainless steel. Stress is incrementally applied to the steel and, just prior to the metal deforming plastically, decrementally released. It is provided for comparison to curves A and B.

In one embodiment of the present invention, stent 10 material includes a cold formed nickel-titanium alloy. Linear pseudoelasticity behavior for a nickel-titanium alloy can result, for example, by using a cold working processing method. The material used to form stent 10 can contain about 30 percent to about 60 percent cold working when measured by the reduction in cross-sectional area. The cold worked percentage can be calculated by the following equation:

$$CW \% = \frac{{S}_{o} - {S}_{i}}{{S}_{o}} \times 100$$

where Sₒ is the initial cross-sectional area of the material before the cold working, and Sᵢ is the cross-sectional material after cold working. There is substantially no heat treatment following the cold working process. Non-linear pseudoelasticity behavior for nickel-titanium alloy can result from cold working and subsequent heat treatment. By limiting the processing parameters to cold working, the nickel-titanium alloy used to form the structural members of stent 10 are in a martensitic phase when the body is stressed into a first shape (e.g., a collapsed form) and also when the stress on the body is relieved to assume a second shape (e.g., an expanded form).

The nickel-titanium alloy can have about 49 atomic percent to about 51 atomic percent nickel, with the remaining material being titanium. The nickel-titanium alloy can also contain a ternary element such as palladium, platinum, chromium, niobium, rhodium, iron, cobalt, vanadium, manganese, boron, copper, aluminum, tungsten, tantalum, or zirconium.

In one embodiment, the nickel-titanium alloy has a transformation temperature set above a typical human body temperature of 37° C. The transformation temperature can be measured by the austenite finish temperature (A_{f}). Other transformation temperatures such as the austenite start temperature (Aₛ), the martensite start temperature, (Mₛ), or the martensite finish temperature (M_{f}) can also be used as the defining metric. It is understood that the austenite finish temperature (A_{f}) is defined to mean the temperature at which the material completely reverts to austenite. The A_{f} is ideally determined by a Differential Scanning Calorimeter (DSC) test, known in the art. The DSC test method to determine transformation temperatures for a nickel-titanium alloy ingot is guided by ASTM standard No. F2004-00, entitled “Standard Test Method for Transformation Temperature of Nickel-Titanium Alloys by Thermal Analysis,” or by an equivalent test method known in the art.

Alternatively, the “active Aₜ” for a tubing used to manufacture stent 10 or other implantable medical device is determined by a bend and free recovery test, also known in the art. In such a test, the tubing is cooled to under the M_{f} temperature, deformed, and warmed up.

While monitoring the increasing temperature, the point of final recovery of the deformation in the tubing approximates the A_{f} of the material. The active A_{f} testing technique is guided by ASTM standard No. F2028-01, entitled “Standard Test Method for Determination of Transformation Temperature of Nickel-Titanium Shape Memory Alloys by Bend and Free Recovery,” or by an equivalent test method known in the art.

The Aₛ or A_{f} of a nickel-titanium alloy material can be adjusted by various methods known in the art. For example, changing the ratio of nickel to titanium, cold working the material, use of a ternary or a quaternary element, all affect the transformation temperature. Accordingly, when stent 10 is used within a mammalian body which is approximately 37° C., having the Aₛ or A_{f} set above that body temperature insures that the device remains in the martensitic phase throughout its use within the body. Insofar as the martensitic phase is maintained as described, the nickel-titanium alloy exhibits only linear pseudoelasticity when the medical device encounters operating stresses. Thus, by operating in this martensitic range, it is possible to exploit the beneficial properties of a linear pseudoelastic nickel-titanium alloy. These beneficial properties are described elsewhere, but include much greater reversible strain as compared to a stainless steel, and greater strength as compared to operating in a non-linear pseudoelastic range.

As mentioned above, in an embodiment of the present invention, a polymeric coating is disposed over a portion of the metallic body of stent 10. The composition for the coating can include a solvent, a polymer dissolved in the solvent and optionally a therapeutic substance. The composition can be applied to the surface of stent 10 by any conventional means, such as spraying or dipping, and a final heat treatment can be conducted to remove essentially all of the solvent from the composition to form the coating.

Representative examples of polymers that can be used to coat a stent in accordance with the present invention include ethylene vinyl alcohol copolymer (commonly known by the generic name EVOH or by the trade name EVAL), poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether-esters) (e.g. PEO/PLA); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; polybutylmethacrylate; rayon; rayon-triacetate; poly(glycerol-sebacate); cellulose acetate; cellulose butyrate; cellulose acetate butyrate; cellophane; cellulose nitrate; cellulose propionate; cellulose ethers; and carboxymethyl cellulose.

“Solvent” is a liquid substance or composition that is compatible with the polymer and is capable of dissolving the polymer at the concentration desired in the composition. Representative examples of solvents include chloroform, acetone, water (buffered saline), dimethylsulfoxide (DMSO), propylene glycol methyl ether (PM,) iso-propylalcohol (IPA), n-propylalcohol, methanol, ethanol, tetrahydrofuran (THF), dimethylformamide (DMF), dimethyl acetamide (DMAC), benzene, toluene, xylene, hexane, cyclohexane, heptane, octane, pentane, nonane, decane, decalin, ethyl acetate, butyl acetate, isobutyl acetate, isopropyl acetate, butanol, diacetone alcohol, benzyl alcohol, 2-butanone, cyclohexanone, dioxane, methylene chloride, carbon tetrachloride, tetrachloroethylene, tetrachloro ethane, chlorobenzene, 1,1,1-trichloroethane, formamide, hexafluoroisopropanol, 1,1,1-trifluoroethanol, and hexamethyl phosphoramide and a combination thereof.

The therapeutic substance contained in the coating can be for inhibiting the activity of vascular smooth muscle cells. More specifically, the therapeutic substance can be aimed at inhibiting abnormal or inappropriate migration and/or proliferation of smooth muscle cells for the inhibition of restenosis. The therapeutic substance can also include any active agent capable of exerting a therapeutic or prophylactic effect in the practice of the present invention. For example, the therapeutic substance can be for enhancing wound healing in a vascular site or improving the structural and elastic properties of the vascular site. Examples of therapeutic substances include antiproliferative substances such as actinomycin D, or derivatives and analogs thereof (manufactured by Sigma-Aldrich 1001 West Saint Paul Avenue, Milwaukee, Wis. 53233; or COSMEGEN available from Merck). Synonyms of actinomycin D include dactinomycin, actinomycin IV, actinomycin I₁, actinomycin X₁, and actinomycin C₁. The therapeutic substance can also fall under the genus of antineoplastic, antiinflammatory, antiplatelet, anticoagulant, antifibrin, antithrombin, antimitotic, antibiotic, antiallergic and antioxidant substances. Examples of such antineoplastics and/or antimitotics include paclitaxel (e.g. TAXOL® by Bristol-Myers Squibb Co., Stamford, Conn.), docetaxel (e.g. Taxotere®, from Aventis S.A., Frankfurt, Germany), methotrexate, azathioprine, vincristine, vinblastine, fluorouracil, doxorubicin hydrochloride (e.g. Adriamycin® from Pharmacia & Upjohn, Peapack, N.J.), and mitomycin (e.g. Mutamycin® from Bristol-Myers Squibb Co., Stamford, Conn.) Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™ (Biogen, Inc., Cambridge, Mass.) Examples of such cytostatic or antiproliferative agents include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents which may be appropriate include alpha-interferon, genetically engineered epithelial cells, tacrolimus, dexamethasone, and rapamycin and structural derivatives or functional analogs thereof, such as 40-O-(2-hydroxy)ethyl-rapamycin (known by the trade name of EVEROLIMUS available from Novartis), 40-O-(3-hydroxy)propyl-rapamycin, 40-O-[2-(2-hydroxy)ethoxy]ethyl-rapamycin, and 40-O-tetrazole-rapamycin.

The dosage or concentration of the therapeutic substance required to produce a favorable therapeutic effect should be less than the level at which the therapeutic substance produces toxic effects and greater than the level at which non-therapeutic results are obtained. The dosage or concentration of the therapeutic substance required to inhibit the desired cellular activity of the vascular region can depend upon factors such as the particular circumstances of the patient; the nature of the trauma; the nature of the therapy desired; the time over which the ingredient administered resides at the vascular site; and if other therapeutic substances are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

### Method of Forming the Implantable Medical Device

The method of forming stent 10 can begin with providing a linear pseudoelastic material that is to be coated with the polymeric composition. For example, a cold worked nickel-titanium alloy can be obtained from Fort Wayne Metals, Fort Wayne, Ind. Such cold worked nickel-titanium alloys can be prepared by using a cold work process. For instance, a nickel-titanium alloy tube can be provided (available from Minitubes, Grenoble, France) and can be placed on a hard mandrel (available from Minitubes) and then subjected to sufficient pressure to reduce the thickness of the wall of the tube to a selected thickness. A heat sink such as a cold water bath can be used to prevent the material from being exposed to heat produced by the process. The cold worked percentage can be calculated by comparing the cross-sectional area of the material before and after the cold working process.

The outer diameter of the tube provided can be about the same diameter as the target lumen. For example, the outer diameter of the tube can be about 1 mm greater than the diameter of the targeted biological lumen. Therefore, as stent 10 is deployed in the expanded state in the biological lumen, the outer surface of stent 10 can sufficiently press against the targeted tissue surrounding the biological lumen.

Once a metallic substrate is provided having a linear pseudoelastic material, the pattern of the structural members of stent 10 can be formed. For example, the structural members of stent 10 can be formed by selectively removing portions of the tube by processes that do not expose the material to high temperatures (e.g., about greater than 300° C.) that can cause the material to lose its linear pseudoelastic behavior. A representative example of a method of forming the structural members includes using a chemical etching process. Chemical etching is a manufacturing technique whereby selected portions of a metal surface are blanked or dissolved away using a chemical etchant or an acid(s). The desired placement of the structural elements can be performed by physically protecting portions of the stent material, for example, by using a mask that is not substantially dissolved by the chemical etchant or acid. A representative example of a suitable etchant includes hydrofluoric acid. Representative examples of suitable materials to be used as masks include stainless steel, aluminum, brass, bronze, polymers, glass, and ceramic. The number of structural elements (e.g., struts 18) can be any number positioned in any suitable configuration which provides sufficient expandability within the biological lumen to properly deploy and maintain stent 10 in place. Likewise, the particular size and shape of each structural element can be varied.

Another representative example of a method of forming structural elements of stent 10 includes using a low-energy laser to cut away portions of the tubular substrate. The low-energy laser can form the struts with out exposing the material to high temperatures. An example of a low-energy laser is the Microjet® (available from Synova SA, Lausanne, Switzerland) which is a water jet-laser hybrid.

Depending on the process used to form the structural members of stent 10, it may be useful to subject the structural members to an electropolishing or descaling process subsequent to their formation. The electropolishing or descaling process, for instance, can be used to remove portions of the metallic substrate that may have been exposed to heat during the process of forming the structural members. In other words, the electropolishing or descaling process can be used to remove zones of the substrate that can no longer exhibit linear pseudoelastic behavior due to exposure to excessive heat. A representative example of a substance that can be used for a descaling process is aqua regia, which is a mixture of concentrated hydrochloric acid (HCl) and nitric acid (HNO₃), containing one part by volume of HNO₃ and three parts of HCl. The heat affected zones can also be removed by a physical descaling process such as using bead blasting.

After the structural members of stent 10 are formed having the linear pseudoelastic material, the polymeric coating can be applied to stent 10. Various methods can be used to apply the coating such as dipping and spraying. The following method of application is being provided by way of illustration and is not intended to limit the embodiments of the present invention. A spray apparatus, such as EFD 780S spray device with VALVEMATE 7040 control system (manufactured by EFD Inc., East Providence, R.I.), can be used to apply a composition to stent 10. EFD 780S spray device is an air-assisted external mixing atomizer. The composition is atomized into small droplets by air and uniformly applied to the stent surfaces. The atomization pressure can be maintained at a range of about 5 psi to about 20 psi. The droplet size depends on such factors as viscosity of the solution, surface tension of the solvent, and atomization pressure. Other types of spray applicators, including air-assisted internal mixing atomizers and ultrasonic applicators, can also be used for the application of the composition.

Each repetition of the spraying process can be followed by removal of a significant amount of the solvent(s). Depending on the volatility of the particular solvent employed, the solvent can evaporate essentially upon contact with stent 10. Alternatively, removal of the solvent can be induced by baking the stent in an oven at a mild temperature (e.g., 60° C.) for a suitable duration of time (e.g., 2-4 hours) or by the application of warm air. Any suitable number of repetitions of applying the composition followed by removing the solvent(s) can be performed to form a coating of a desired thickness or weight.

Subsequent to the application of the composition to stent 10 and the formation of the polymeric coating, stent 10 can be integrated with a stent delivery system. A mechanical apparatus can be used to collapse stent 10 to a size useful for deployment on a delivery system, for example, a system using a catheter apparatus. For example, the mechanical apparatus can apply sufficient radial force to collapse the body of stent 10. This mechanical method can be used as an alternative to a method that subjects a stent to a sufficiently low temperature that causes temperature-induced martensite and hence collapse of the stent. For example, in a particular method, liquid nitrogen is used to collapse a polymer coated stent. Subjecting a stent with a polymer coating to such low temperatures can make the polymer of the coating very brittle and hence the coating susceptible to cracking during stress. Stent 10, however, does not undergo phase transformation at room temperature when the body is subjected to stress used to collapse the body of stent 10.

After stent 10 is collapsed, stent 10 can be inserted into a sheath that at least partially envelops the body of stent 10 in the collapsed state. The restraining sheath can have sufficient elasticity to resist the outward bias of struts 18. One manner of achieving the required elasticity is through selection of a particular size and wall thickness for the sheath. Another is through use of an elastic material that has sufficient resilience to resist the expansive forces of struts 18. Such sheath materials and designs are known in the art.

The sheath may be used to transport the device to a targeted location in the patient's anatomy and to deploy the device. Once stent 10 is transported to the targeted location, the sheath is removed so that struts 18 expand to the expanded state because struts 18 have a radially outward bias toward the expanded position.

### Method of Use

A drug can be applied to a stent, retained on the stent during delivery and expansion of the stent, and released at a desired rate and for a predetermined duration of time at the site of implantation. A stent having the above-described coating is useful for a variety of medical procedures, including, by way of example, treatment of obstructions caused by tumors in bile ducts, esophagus, trachea/bronchi and other biological passageways. A stent having the above-described coating is particularly useful for treating occluded regions of blood vessels caused by abnormal or inappropriate migration and proliferation of smooth muscle cells, thrombosis, and restenosis. Stents may be placed in a wide array of blood vessels, both arteries and veins. Representative examples of sites include the iliac, renal, and coronary arteries.

Briefly, an angiogram is first performed to determine the appropriate positioning for stent therapy. An angiogram is typically accomplished by injecting a radiopaque contrasting agent through a catheter inserted into an artery or vein as an x-ray is taken. A guidewire is then advanced through the lesion or proposed site of treatment. Over the guidewire is passed a delivery catheter which allows a stent in its collapsed configuration to be inserted into the passageway. The delivery catheter is inserted either percutaneously or by surgery into the femoral artery, brachial artery, femoral vein, or brachial vein, and advanced into the appropriate blood vessel by steering the catheter through the vascular system under fluoroscopic guidance. A stent having the above-described coating may then be expanded at the desired area of treatment by removing a restraining sheath. A post-insertion angiogram may also be utilized to confirm appropriate positioning.

### EXAMPLE

The embodiments of the invention will be illustrated by the following set forth example which is being given by way of illustration only and not by way of limitation. All parameters and data are not be construed to unduly limit the scope of the embodiments of the invention.

### Example 1

The following study was performed to determine the effect of heat exposure on a cold-worked nickel-titanium alloy. Cold-worked nitinol wires were provided by Fort Wayne Metals. Two sets of the wires had been cold worked to provide cold work percentages of 35.6% and 49.7%. The following equipment was used for the study: (1) Instron® Model 5565 (available from Instron Corporation, Canton, Mass.); (2) a Load Cell (1000 lbs); (3) Wedge Action Grips (30 kN); (4) Video Extensometer with 100 mm FOV lens; and (5) Thermolyne® 2110 tube furnace. The control group was not exposed to heat treatment, and another test group was exposed to 400° C. for 2 minutes. The test groups were then subjected to a tensile test using the Instron® machine.

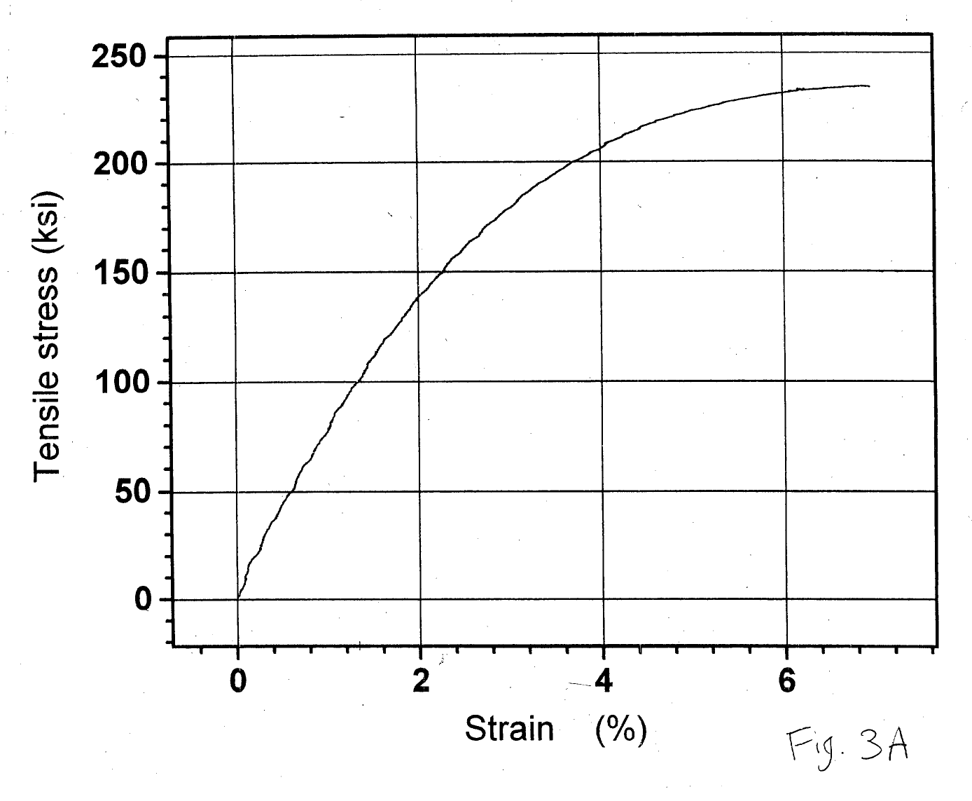

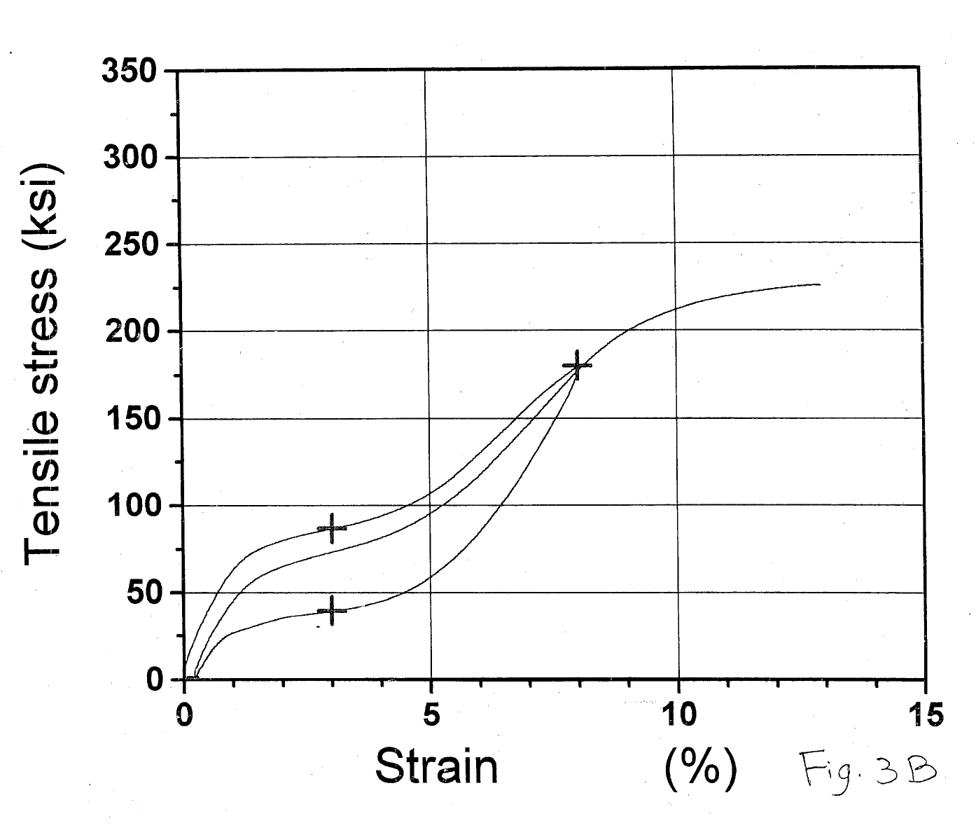

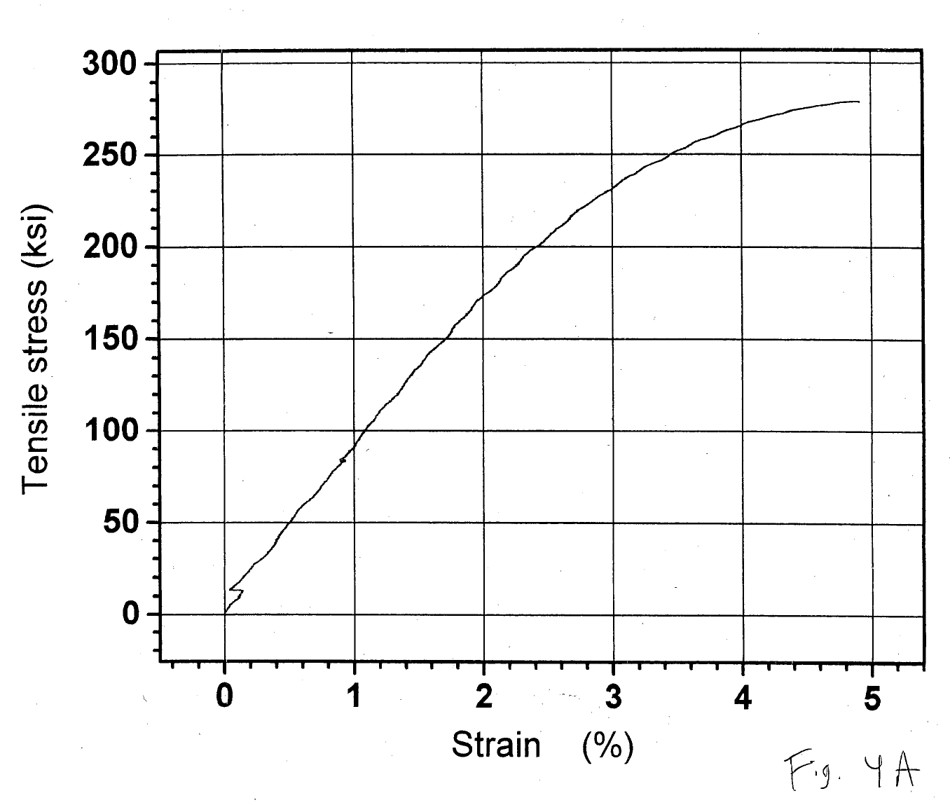

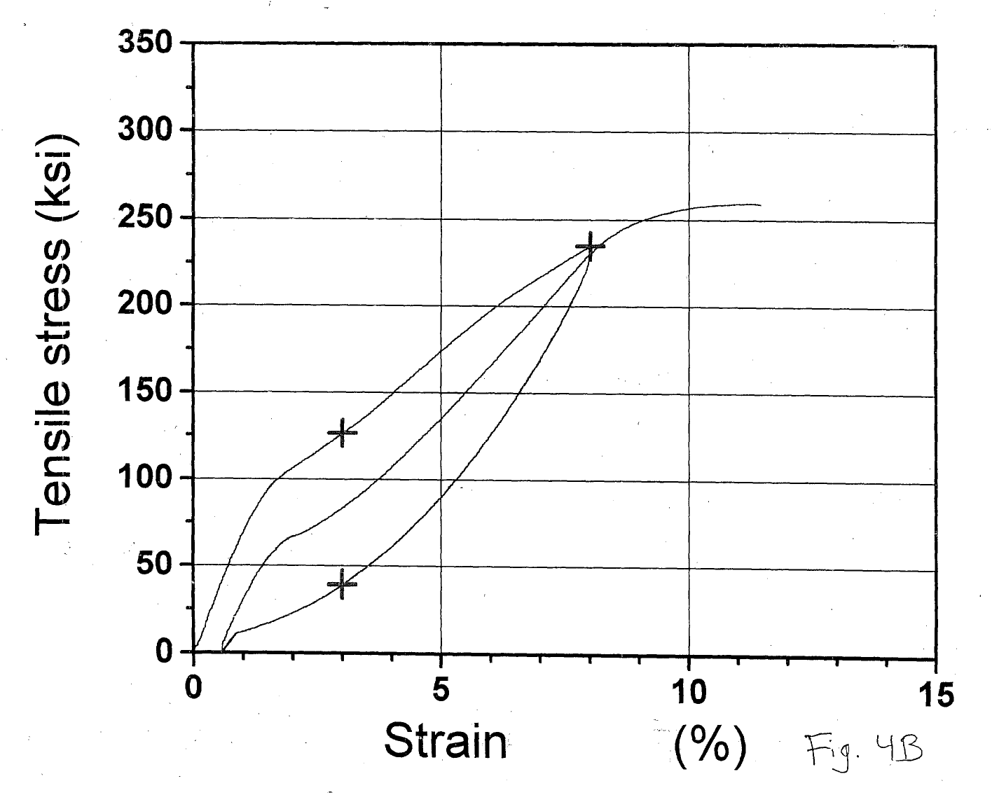

The results of the test showed that the wires that were exposed to heat treatment exceeding particular temperatures no longer exhibited linear pseudoelastic behavior. For instance, for the wires having a cold worked percentage of 35.6%, a comparison of FIGS. 3A (no heat treatment) and 3B (400° C.) demonstrates that these wires lost their linear pseudoelastic behavior after being exposed to a heat treatment of 400° C. Additionally, for the wires having a cold worked percentage of 49.7%, a comparison of FIGS. 4A (no heat treatment) and 4B (400° C.) demonstrates that these wires lost their linear pseudoelastic behavior after being exposed to a heat treatment of 400° C. The results also showed that the wires having a greater cold work percentage were able maintain some of their linear pseudoelastic behavior.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. An implantable medical device for insertion into a biological lumen, comprising:
a metallic body constructed of a linear pseudoelastic material including a ternary element selected from the group consisting of palladium, platinum, chromium, niobium, rhodium, tungsten, tantalum, and zirconium; and
a coating comprising a polymer disposed over a portion of the body, wherein the coating further comprises a therapeutic substance.

2. The implantable medical device of claim 1, wherein the linear pseudoelastic material does not undergo phase transformation when the body is subjected to stress.

3. (canceled)

4. An implantable medical device for insertion into a biological lumen, comprising;
a metallic substrate; and
a coating comprising a polymer disposed over a portion of the substrate, wherein the substrate is in a martensitic phase when the substrate is stressed into a first shape and the substrate remains in a martensitic phase when the stress on the substrate is relieved to assume a second shape, and wherein a stress-strain hysteresis curve for the substrate does not include a stress plateau, and wherein the substrate includes a ternary element selected from the group consisting of palladium, platinum, chromium, niobium, rhodium, tungsten, tantalum, and zirconium.

5. (canceled)

6. A stent for insertion into a biological lumen, comprising:
a self-expanding body comprising a cold formed nickel-titanium alloy, the self-expanding body including a plurality of laser-cut struts that have been physically descaled so that substantially all of the cold formed nickel-titanium alloy exhibits linear pseudoelastic behavior and substantially none of the cold formed nickel-titanium alloy exhibits nonlinear pseudoelastic behavior; and
a coating disposed over a portion of the body, the coating comprising a polymer and a therapeutic substance.

7. The stent of claim 6, wherein the cold formed nickel-titanium alloy comprises a cold worked percentage of about 30% to about 60%.

8. (canceled)

9. (canceled)

10. The stent of claim 6, wherein the alloy has a transformation temperature greater than a mammalian body temperature.

11. The stent of claim 6, wherein the nickel-titanium alloy is pseudo elastic when stressed without onset of stress-induced martensite.

12.-15. (canceled)

16. A method of producing the implantable medical device of claim 1, comprising:
forming struts by selectively removing portions of the metallic body, the metallic body being a tubular substrate, the substrate comprising a cold worked metallic material; and
applying the coating to the struts, the coating comprising the polymer and a therapeutic sub stance.

17. The method of claim 16, wherein when stress is applied to the coated struts to place the struts into a compressed form, the substrate exhibits linear pseudo elasticity during the application of the stress.

18. The method of claim 17, wherein the application of stress does not create stress-induced martensite in the substrate.

19. The method of claim 16, wherein the formation of the struts is performed by using a low-energy laser.

20. The method of claim 16, wherein the formation of the struts is performed by chemical etching.

21. The device of claim 1, wherein the biological lumen is a vascular lumen.

22. The device of claim 4, wherein the coating further comprises a therapeutic agent.

23. The device of claim 4, wherein the biological lumen is a vascular lumen.

24. The stent of claim 6, wherein the biological lumen is a vascular lumen.

25. The device of claim 1, wherein the metallic body comprises substantially only the linear pseudoelastic material that is in a martensite phase.

26. A stent for insertion into a biological lumen, comprising:
a self-expanding stent body comprising a cold formed nickel-titanium alloy that exhibits linear pseudoelasticity, the self-expanding stent body including a plurality of struts formed therein in a manner that does not alter the linear pseudoelastic behavior of the cold formed nickel-titanium alloy; and
a coating disposed over at least a portion of the stent body, the coating comprising a polymer and a therapeutic substance.

27. A method of forming the stent of claim 26, comprising:
cutting the plurality of struts in a body constructed of the cold formed nickel-titanium alloy that exhibits the linear pseudoelastic behavior so that the linear pseudoelastic behavior of the cold formed nickel-titanium alloy is not substantially altered.

28. A stent, comprising:
a self-expanding stent body including a plurality of laser-cut struts, the self-expanding stent body constructed of a cold formed nickel-titanium alloy, the cold formed nickel-titanium alloy exhibiting linear pseudoelastic behavior at least in heat-affected zones of the self-expanding stent body affected by the laser cutting used to form the plurality of struts; and
a coating disposed over at least a portion of the self-expanding stent body, the coating comprising a polymer and a therapeutic substance.
